(19) United States
(12) Patent Application Publication
(10) Pub. No.: US 2026/0335215 A1
(43) Pub. Date: Oct. 29, 2026

(54) AMPHIPHILIC EMULSION TECHNIQUE FOR TOPICAL REPAIR FORMULA

(71) Applicants: Bonnie D'Arcangelo, Northville, MI (US); Steve D'Arcangelo, Redford, MI (US)

(72) Inventors: Bonnie D'Arcangelo, Northville, MI (US); Steve D'Arcangelo, Redford, MI (US)

(21) Appl. No.: 19/068,962

(22) Filed: Mar. 3, 2025

(51) Int. Cl. A61K 8/73; A61K 8/19; A61K 8/25; A61K 8/49; A61K 8/60; A61K 8/67; A61K 8/92; A61K 8/9789; A61K 8/9794; A61Q 19/00

(52) U.S. Cl. CPC A61K 8/735; A61K 8/19; A61K 8/25; A61K 8/498; A61K 8/60; A61K 8/678; A61K 8/922; A61K 8/925; A61K 8/9789; A61K 8/9794; A61Q 19/00

(57) ABSTRACT

The present invention provides an amphiphilic composition for topical repair, formulated to enhance skin hydration, elasticity, and cellular regeneration. The composition comprises ortho silicic acid, hyaluronic acid, fulvic acid, plant or animalbased oils, and aloe powder. Ortho silicic acid promotes collagen production and cellular repair, while hyaluronic acid functions as a humectant and surfactant, optimizing moisture retention and delivery. Fulvic acid acts as an amphiphilic surfactant, stabilizing the emulsion and enhancing bioavailability. The formulation enables the seamless integration of hydrophilic and lipophilic components, mimicking the human cell's phospholipid bilayer for improved absorption. This innovation supports skin barrier restoration, wound healing, and therapeutic applications for conditions such as neuropathy, varicose veins, and inflammation. The composition can be customized for various dermatological and medicinal uses, providing an effective, stable, and bioavailable system for targeted skin repair and hydration.

### FIELD OF INVENTION

The present invention relates to compositions and formulations for topical and oral applications that provide skin health, hydration, and therapeutic benefits. More specifically, the invention pertains to cosmetic and medicinal preparations comprising Ortho silicic acid, hyaluronic acid, fulvic acid, oil, and aloe powder, which are formulated to enhance skin elasticity, hydration, and overall dermatological health. The invention falls within the technical fields of cosmetic or similar toilet preparations (A61K 8/00), silicon compounds for medicinal or cosmetic use (A61K 31/195), preparations for skin care (A61Q 19/00), and medicinal preparations containing plant based materials (A61K 36/00).

### BACKGROUND OF INVENTION

Skin health and hydration are essential factors in maintaining a youthful and healthy appearance. Over time, exposure to environmental stressors such as UV radiation, pollution, and oxidative stress contributes to skin damage, loss of elasticity, and premature aging. Traditional skincare formulations incorporate moisturizing agents, surfactants, humectants, and emollients to counteract these effects, but many products fail to deliver sustained hydration, enhance skin barrier function, or effectively support cellular repair.

Surfactants play a critical role in numerous industrial, pharmaceutical, and cosmetic applications. Surfactants are substances that lower surface tension between two liquids, a liquid and a gas, or a liquid and a solid. These compounds enhance solubility and facilitate interactions between hydrophobic and hydrophilic substances. Typically, a surfactant is an organic compound composed of a hydrophilic portion and a hydrophobic portion. The hydrophilic portion is often referred to as the “head”, while the hydrophobic portion is often referred to as a “tail.” The head of a surfactant is usually polar, although they do not necessarily carry a charge. The tail is typically a nonpolar hydrocarbon chain. Traditional surfactants, however, often exhibit limitations such as environmental persistence, toxicity, and instability under certain conditions. The need for more efficient, biodegradable, and structurally versatile surfactants continues to drive research and innovation in the field of cosmetics.

Humectants are common ingredients in a wide range of cosmetic and personal care products that are intended to moisturize or retain water. A humectant is a hygroscopic substance used to reduce the loss of water. In pharmaceuticals and cosmetics, humectants increase the solubility of a chemical compound's active ingredients, increasing the active ingredients' ability to penetrate skin, or its activity time. This hydrating property also counteracts any dehydrating active ingredient effect a composition may have.

Current cosmetic formulations rely on a limited range of active ingredients that often provide temporary hydration without addressing underlying structural deficiencies in the skin. Many hydrating compounds lack the ability to penetrate deeply into the skin or degrade rapidly upon exposure to air and light. Additionally, conventional formulations struggle to balance bioavailability, stability, and efficacy, limiting their overall therapeutic impact.

Current amphiphilic compounds rely on conventional molecular designs, often resulting in limited functionality and adaptability. Many existing surfactants struggle to maintain performance across varying pH levels, temperatures, and ionic strengths. Additionally, achieving precise control over self-assembly properties and interfacial behavior remains a challenge. These limitations hinder their applicability in fields such as drug delivery and cosmetics.

The present invention addresses these challenges by providing an optimized composition that enhances skin hydration, strengthens the extracellular matrix, and promotes overall skin repair. Through a carefully balanced formulation of Ortho silicic acid, hyaluronic acid, fulvic acid, aloe powder, and oil, the disclosed invention delivers superior bioavailability, stability, and therapeutic benefits. This novel approach offers improved cosmetic and dermatological applications for anti-aging, hydration, and skin barrier restoration.

### SUMMARY OF THE INVENTION

This summary is intended to disclose the present invention, an amphiphilic emulsion technique for topical repair composition. Embodiments of the invention are presented to illustrate and inform one skilled in the art.

The present invention is patentable subject matter that has not been disclosed or taught in any prior art reference. This summary outlines representative features of the amphiphilic emulsion technique for topical repair composition in order that the detailed description may be better understood. There are additional design elements and embodiments of the amphiphilic emulsion technique for topical repair composition that will be described and that will be incorporated into the claims in this application.

The embodiments described in this summary are intended to illustrate the scope of the subject matter. The embodiments do not represent all of the embodiments possible under the scope of the subject matter. They are intended to be illustrative. The amphiphilic emulsion technique for topical repair composition is capable of other embodiments and of being practiced and carried out in various ways. Also, the phraseology and terminology employed herein are for the purpose of the description and are not limiting.

The present invention is an amphiphilic emulsion technique for topical repair composition comprised of Ortho silicic acid, hyaluronic acid, fulvic acid, oil, and aloe powder. The present invention has demonstrated significant potential in improving skin hydration, elasticity, and repair. Ortho silicic acid promotes collagen production, which helps improve skin elasticity, firmness, and hydration. Typically, hyaluronic acid is a hydrophilic humectant that retains moisture. It can also be modified to act as a surfactant. Fulvic acid is an amphiphilic surfactant that exhibits antioxidant and anti-inflammatory properties. Current formulations incorporating certain of these ingredients often suffer from instability, poor absorption, or ineffective synergistic interactions. The composition herein disclosed overcomes these issues.

This invention concerns the use of an amphiphilic emulsion technique to create a topical repair composition. An amphiphilic emulsion technique for a topical repair composition uses molecules that have both water-loving (hydrophilic) and oil-loving (lipophilic) properties to create stable mixtures. This method actively blends water-based and oil-based ingredients into a single, effective emulsion. The amphiphilic molecules organize themselves at the interface between the two phases, which not only stabilizes the formulation but also enhances the delivery of active ingredients to the skin. This technique promotes effective absorption, supports the skin's natural barrier, and accelerates repair processes, making it ideal for wound care and skin recovery applications.

In practice, amphiphilic molecules act as efficient carriers, helping hydrophilic and lipophilic ingredients blend seamlessly into a stable formulation. They penetrate the skin more effectively, ensuring deeper delivery of healing compounds. This method improves hydration, reinforces the skin barrier, and accelerates the repair process.

### BRIEF DESCRIPTION OF THE DRAWINGS

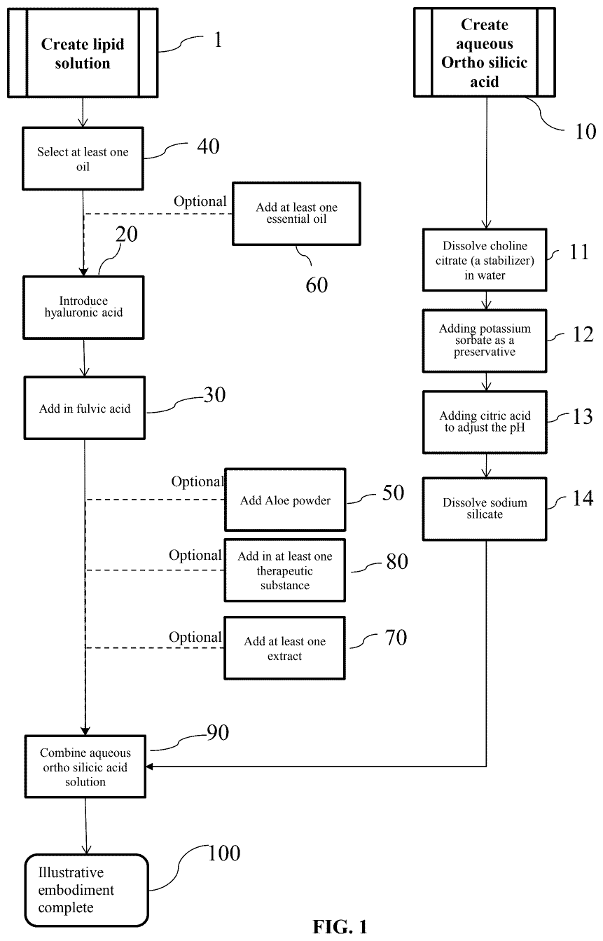

The present invention is illustrated with 1 drawing on 1 sheet. The accompanying drawing, FIG. 1, which are incorporated in and constitute a part of this disclosure, is a flow chart that illustrates the method by which the composition of the present invention is formulated.

### DETAILED DESCRIPTION

The following descriptions are not meant to limit the invention, but rather to add to the summary of invention, and illustrate the present invention, an amphiphilic emulsion technique for topical repair composition. The present invention is illustrated with a drawing showing the primary method used in the present invention's formation.

Certain terminology is used in the following description for convenience only and is not limiting. The article “a” is intended to include one or more items, and where only one item is intended the term “one” or similar language is used. To assist in the description of the present invention, words such as short, long, top, bottom, side, upper, lower, front, rear, inner, outer, right and left are used to describe the relative size and orientation of the present invention, an amphiphilic emulsion technique for topical repair composition, with respect to the accompanying. The terminology includes the words above specifically mentioned, derivatives thereof, and words of similar import.

The following detailed description refers to the accompanying drawing. Wherever possible, the same reference number is used across the descriptions of the drawing and embodiments to refer to the same or similar elements. The embodiments of the claimed subject matter may be described, modified, and adapted, and other implementations are possible. For example, substitutions, additions, or modifications, which, perform identical functions to the embodiments disclosed, may be made to the elements illustrated in the drawings. Accordingly, the following detailed description does not limit the claimed subject matter. The proper scope of the claimed subject matter is defined by the claims contained herein. The claimed subject matter improves over the prior art by providing an amphiphilic emulsion technique for topical repair composition.

Embodiments described above with reference to functions or acts, comprise methods. The functions/acts noted above may occur out of the order as shown or described. For example, two functions/acts shown or described in succession may in fact be executed substantially concurrently or the functions/acts may sometimes be executed in the reverse order, depending upon the functionality/acts involved. While certain embodiments have been described, other embodiments may exist. Further, the disclosed methods' functions/acts may be modified in any manner, including by reordering functions/acts and/or inserting or deleting functions/acts, without departing from the spirit of the claimed subject matter.

Although the subject matter has been described in language specific to structural features and/or methodological acts of the illustrated figures, it is to be understood that the subject matter defined in the appended claims is not necessarily limited to the specific features or acts described above. Rather, the specific features and acts described above are disclosed as example forms of implementing the claims.

A Dalton (Da) is a unit of mass commonly used to express the mass of molecules, particularly in the fields of biochemistry and molecular biology. It is defined as one-twelfth of the mass of a carbon-12 atom, at its lowest energy state, which is approximately 1.66053906660×10\hat{ }-24 grams. The Dalton is synonymous with the unified atomic mass unit (u), making it a convenient measure for describing atomic and molecular weights. In this application, the Dalton is used to quantify the mass of various molecules such as lipids, proteins, acids, oils, and other large macromolecules.

The present invention is a composition containing ortho silicic acid which is intended for topical application, and which penetrates through the skin barrier and provides cellular repair. The exemplary embodiment of the present invention comprises a formulation containing Ortho silicic acid, hyaluronic acid, fulvic acid, plant-or animal-based oils, and aloe powder. The present invention improves the healing process for various types of tissue, including skin, muscle, and cartilage, by providing a moisture-rich environment and delivering targeted substances that stimulate cellular repair and regeneration. The ability of the invention to hold both the hydrophilic and hydrophobic substances in solution allows for delivery in a single application. This amphiphilic solution allows for advance delivery of the ingredients, mimicking the human body's cellular structure. The addition of variations of the invention includes other ingredients that increase the potential of the base invention to further deliver repair to muscle, cartilage, nerves, and tissue.

The amphiphilic emulsion technique disclosed in this patent represents a specific method of emulsification and dispersion to create a stable and effective topical composition. This process allows both oil-soluble (lipophilic) and water-soluble (hydrophilic) components to exist in a single composition, enhancing delivery and absorption into the skin and body. This amphiphilic composition allows for advance delivery of the ingredients, mimicking the human body's cellular structure.

Ortho silicic acid promotes cellular repair, stimulates healthy tissue growth, and is small enough (96 Daltons) that it can cross the blood-brain barrier. Ortho Silicic Acid is synthesized by combining sodium silicate, citric acid, choline citrate, and potassium sorbate. The choline citrate stabilizes the molecular bond of the solution, while potassium sorbate serves as a preservative. This form of silicic acid can be used in various soluble forms, including water-soluble, non-water-soluble, and oil-soluble forms, as well as in both choline-stabilized and non-choline-stabilized variants. The composition is not limited to a specific preparation but is adaptable to different forms of ortho silicic acid.

Hyaluronic acid modifies the body's response to tissue damage, promoting wound healing, scar reduction, and enhanced hydration to tissues. Sodium hyaluronate (SH) is the sodium salt of hyaluronic acid (HA), a polysaccharide composed of d-glucuronic acid and N-acetylglucosamine. The biological effects of hyaluronic acid depend heavily on molecular weight, which is measured on the Dalton scale. Hyaluronic acid with molecular weights from 400 to 4000 Daltons (0.4-4 kDa) acts as an inducer of heat shock proteins and has a non-apoptotic property. Polysaccharides with a molecular weight equal to 6-20 kDa possess immunostimulatory, angiogenic, and phlogotic activities. Hyaluronic acid with a molecular weight of 20-200 kDa takes part in biological processes such as embryonic development, wound healing and ovulation. By contrast, high molecular weight hyaluronic acid (>500 kDa) has anti-angiogenic activity, and can function as a space filler and a natural immunologic depressant. The largest molecular weight of hyaluronic acid in the human body is approximately 20,000 kDa.

In the disclosed composition, hyaluronic acid serves as both a humectant and a surfactant. It is used to facilitate the emulsification of oil and water components by binding to the lipophilic tails of the oil (e.g., olive oil), allowing for better interaction with hydrophilic components such as ortho silicic acid. For the formulation, one should use a combination of a low molecular weight hyaluronic acid and a high molecular weight hyaluronic acid. A combination of low and high molecular structures of sodium hyaluronate delivers both surface and deep tissue hydration. The formulation includes sodium hyaluronate in a powdered form, with a molecular weight ranging from 1 kDa to 20,000 kDa. Hyaluronic acid should be selected so that the low molecular weight is in the range of 3 kDa to 7 kDa and the high molecular weight hyaluronic acid is in the range from 10,000 kDa to 20,000 kDa. In the illustrative embodiment, a ratio for the hyaluronic acid components is 5 parts of 5 kDa Hyaluronic Acid to 10 parts of 20,000 kDa Hyaluronic Acid, though variations in the molecular weight ratio are possible.

Fulvic acid acts as an amphiphilic surfactant, effectively emulsifying oil and water components to keep them in suspension. Additionally, fulvic acid possesses healing properties, reducing inflammation and serving as an antioxidant. Its short-chain Dalton structure aids in the enhanced delivery of active components. Fulvic acid further contributes to the moisture retention of the formulation, supporting tissue hydration and improving overall wound healing rates. The disclosed topical composition uses commercially available fulvic acid with a clean certificate of analysis.

The composition includes commercially available oils in all forms. In the illustrative embodiment, at least one plant- or animal-based oil, selected from a variety of plant- or animal-based oils, to provide moisture and improve skin repair. Oils such as Olive oil, Evening Primrose oil, Sesame oil, Blackseed oil, Currant seed oil, Pomegranate oil, Almond oil, Macadamia oil, Flax seed oil, Sea Buckthorn oil, Coconut oil, Grapeseed oil, and others can be used, each contributing unique benefits. The oils assist in forming the lipid phase of the formulation, offering enhanced hydration and nourishing properties to the skin. These oils may also help facilitate the absorption of other active ingredients into the skin.

Aloe powder is derived from dehydrated aloe gel and contributes to the moisturizing and healing properties of the formulation. Aloe has been known for its ability to soothe the skin, reduce inflammation, and accelerate wound healing.

Referring now to FIG. 1, the emulsion formation is achieved by separately creating a lipophilic phase 1 and hydrophilic phase 10 and then combining them 90 into the illustrative embodiment of the present invention 100. The core of the amphiphilic emulsion technique is its emulsion system, which disperses oil molecules within a water-based solution. This is achieved using fulvic acid and hyaluronic acid as amphiphilic agents.

First, a hydrophilic phase, comprised of an ortho silicic acid solution, is created 10. Choline citrate, a molecular stabilizer, is dissolved in water 11. The choline citrate is stirred until is dissolved into a solution. Potassium sorbate is added as a preservative 12 and it is stirred until completely dissolved in the solution. Citric acid is added to the solution to adjust the pH 13 and it is stirred until it is completely dissolved into the solution. Last, sodium silicate is dissolved into the aqueous solution 14. This solution serves as the hydrophilic phase.

A lipophilic phase is created by selecting one or more oils 40. In the illustrative embodiment, the oil is a plant-or animal-based oil. The plant-or animal-based oils include, but are not limited to, Olive oil, Evening Primrose oil, Sesame oil, Blackseed oil, Currant seed oil, Pomegranate oil, Almond oil, Macadamia oil, Flax seed oil, Sea Buckthorn oil, Coconut oil, Grapeseed oil, or Emu oil.

One or more essential oils may optionally be added 60 to the at least one oil 40. The essential oils 60 may include, but are not limited to, Lavandula Augustifolia essential oil, Eucalyptus essential oil, Rosmarinus officinalis (aka Rosemary) essential oil, Myrrh gum essential oil, Juniper essential oil, Cypress essential oil, Bergamot essential oil, Orange essential oil, Frankincense essential oil, Turmeric essential oil, Ginger essential oil, Neroli essential oil, Chamomile essential oil, Marjoram essential oil, Marjoram essential oil, and Lemon essential oil.

Hyaluronic acid is introduced into the oil as powdered sodium hyaluronate 20, and it is stirred until blended and completely dissolved. Both low molecular weight (5 kDa) and high molecular weight (20,000 kDa) sodium hyaluronate are used, in a 5:10 ratio. A combination of low and high molecular weight hyaluronic acid ensures hydration at different levels of skin tissue. Hyaluronic acid binds to the oil molecules'hydrophobic tails, helping them disperse in the aqueous solution.

Fulvic acid is added to the lipophilic solution 30 and it is stirred until blended and completely dissolved. Fulvic acid is naturally amphiphilic, meaning it can bind both oil and water molecules. It stabilizes the emulsion, keeping the oil in suspension rather than allowing it to separate. It also enhances penetration of the active ingredients by reducing inflammation and acting as an antioxidant.

The hydrophobic components (oils) are dispersed evenly in the hydrophilic ortho silicic acid solution due to the binding effects of fulvic and hyaluronic acid.

Aloe powder may optionally be added 50. It is stirred until blended and completely dissolved.

Additional therapeutic substances may be optionally added 80. The therapeutic substances may include, but are not limited to, Monoatomic Gold, Orbitally Rearranged Monoetomic Elements, Vitamin E, Crushed or Milled stones precious and non-precious, and Glucosamine Sulfate.

One or more extracts may be optionally added 70. These extracts may include, but are not limited to, Plantago major extract, Calendula officinalis extract, Hamamelis virginiana extract (American Witch-hazel), Pinus Pinaster extract, Ruscus aculeatus extract, Geranium maculatum extract, Gotu Kola (aka Centalla asiatica) extract, Goldenseal extract, and Lemon balm extract.

The aqueous ortho silicic acid 90 solution is combined with the lipid solution 40, 20, 30 to create an amphiphilic composition 100. The amphiphilic composition creates structures similar to the phospholipid bilayer found in human skin cells. This structure allows faster absorption through skin barriers. The composition improves bioavailability and absorption because it mimics the lipid bilayer structure of the human cell, allowing for deeper penetration.

The hydrophilic ingredients (ortho silicic acid) interact with water-based pathways in the skin. Lipophilic ingredients (oils, fatty acids, hyaluronic acid, fulvic acid, and, optionally, aloe) penetrate through fat-based pathways. This dual delivery system ensures that all active ingredients reach their targeted layers effectively.

The molecular size of the constituent ingredients is carefully controlled. In laboratory measurements, the ortho silicic acid was small enough (96 Da) to cross the blood-brain barrier. This low molecular weight (and size) allows the ortho silicic acid to deeply penetrate the skin. The mix of low and high molecular weight hyaluronic acid allows for the small molecules to penetrate the skin deeply to aid in hydration. The large molecule hyaluronic acid retains surface moisture. Short-chain Dalton structure fulvic acid is used to facilitate nutrient delivery and anti-inflammatory action.

The disclosed composition a stable suspension of active ingredients. Unlike traditional oil-based or water-based formulas that require multiple applications for different components, this system holds all ingredients in one solution, ensuring even and controlled delivery.

Each constituent has a therapeutic effect. Moisturizing oils prevent dryness and support wound healing. Hyaluronic acid increases hydration and promotes scar reduction. Ortho silicic acid enhances cell regeneration and stimulates collagen growth. Fulvic acid reduces inflammation and improves nutrient absorption.

This composition allows for application-specific customization that can be optimized for treating diabetic wounds, neuropathy pain, varicose veins, and general skin conditions such as eczema, psoriasis, and burns.

A first alternative embodiment is intended for wound care. The first alternative embodiment is comprised of ortho silicic acid, hyaluronic acid, fulvic acid, olive oil, aloe powder, Plantago major extract, Monoatomic Gold, Lavandula Augustifolia essential oil, Eucalyptus essential oil, Rosmarinus officinalis essential oil, Myrrh gum essential oil, Calendula officinalis extract, and vitamin E. Plantago major is a species of flowering plant in the plantain family Plantaginaceae. Plantago major extract contains phytochemicals including allantoin, aucubin, ursolic acid, flavonoids, and asperuloside. Monoatomic Gold includes the monatomic mineral elements of gold, iridium, rhodium, silver, platinum and chromium. It is often referred to as white gold powder, or the acronyms ORMUS or ORMES (Orbitally Re-arranged Monoatomic Element). This first alternative embodiment of the composition has been found to be therapeutic for diabetic wounds, pressure ulcers, and bed sores.

A second alternative embodiment is intended for the treatment of neuropathy. This second alternative embodiment is comprised of ortho silicic acid, hyaluronic acid, fulvic acid, Aloe powder, Blackseed Oil, Evening Primrose Oil, Monoatomic Gold, Magnesium Citrate, Juniper essential oil, Cypress essential oil, Eucalyptus essential oil, Rosmarinus officinalis essential oil, Bergamot essential oil, Orange essential oil, Myrrh essential oil, Frankincense essential oil, and Turmeric essential oil. This second alternative embodiment of the composition has been found to be therapeutic for neuropathy pain.

A third alternative embodiment is intended for the treatment of varicose veins. This third alternative embodiment is comprised of ortho silicic acid, hyaluronic acid, fulvic acid, Aloe, Blackseed Oil, Evening Primrose Oil, Monoatomic Gold, Aloe, Fulvic Acid, Hamamelis virginiana extract (American Witch-hazel), Pinus Pinaster extract, Ruscus aculeatus extract, Geranium maculatum extract, Gotu Kola extract, Juniper essential oil, Cypress essential oil, Ginger essential oil, Bergamot essential oil, Rosmarinus officinalis essential oil, Neroli essential oil, Myrrh essential oil, Chamomile essential oil, and Turmeric essential oil. This third alternative embodiment of the composition has been found to be therapeutic for veins that are damaged, enlarged, or painful. The formula helps to correct the structure and function of the veins.

A fourth alternative embodiment of the present invention is intended for the treatment of pain and inflammation. This fourth alternative embodiment is comprised of ortho silicic acid, hyaluronic acid, fulvic acid, Aloe, Blackseed Oil, Evening Primrose Oil, Monoatomic Gold, Glucosamine Sulfate, Bergamot essential oil, Ginger essential oil, Neroli essential oil, Marjoram essential oil, Myrrh essential oil, Chamomile essential oil, Turmeric essential oil. This fourth alternative embodiment of the composition has been found to be therapeutic for the relief of pain and inflammation. This composition also provides contains substances, such as Glucosamine Sulfate, which have been proven as effective in the repair of soft tissue at a cellular level.

A fifth alternative embodiment of the present invention is intended for the treatment of pain, inflammation, skin aberrations, eczema, psoriasis, injuries, bruises, minor burns, and other conditions. The fifth alternative embodiment of the composition is comprised of ortho silicic acid, hyaluronic acid, fulvic acid, Aloe, olive oil, Monoatomic Gold, Lemon essential oil, Bergamot essential oil, Frankincense essential oil, and Myrrh essential oil.

The present invention can be practiced without the oil emulsion. A sixth embodiment of the present invention is comprised of ortho silicic acid, hyaluronic acid, fulvic acid, colloidal silver, iodine, Goldenseal extract, Lemon balm extract, Lavender essential oil, Eucalyptus essential oil, Rosmarinus officinalis essential oil, Turmeric essential oil, and Gota Kola extract. This sixth alternative embodiment is intended to treat conditions which require a drying action, such as cold sores, poison ivy and bug bites.

The compositions of the present invention can be practiced with the following non-exclusive list of emulsions: water-in-oil, water-in-oil-in-water, oil-in-water, silicone-in-water, water-in-silicone, oil-in-water-in-oil, and oil-in-water-in-silicone.

It is contemplated that the compositions of the present invention can include any of the substances, or any combination of the substances, described throughout this specification. In particular aspects, the substances are combined. The compositions can include any number of combinations of additional ingredients described throughout this specification. The concentrations of the any ingredient within the compositions can vary. In non-limiting embodiments, for example, the compositions can comprise, consisting essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition. A person of ordinary skill in the art would understand that the concentrations can vary depending on the addition, substitution, and/or subtraction of ingredients in a given composition.

Unless otherwise defined, all technical and scientific terms used herein have the same meaning as commonly understood by one of ordinary skill in the art to which this invention belongs. Although methods and materials similar to or equivalent to those described herein can be used in the practice or testing of the amphiphilic emulsion technique for topical repair composition, suitable methods and materials are described above. All publications, patent applications, patents, and other references mentioned herein are incorporated by reference in their entirety to the extent allowed by applicable law and regulations. The amphiphilic emulsion technique for topical repair composition may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

We claim:

1. A topical amphiphilic composition comprising:
a water-based ortho silicic acid solution;
sodium hyaluronate in powdered form;
fulvic acid; and
oil;
wherein the ortho silicic acid is composed of sodium silicate, citric acid, choline citrate, and potassium sorbate;
wherein the ortho silicic acid is dissolved in water;
wherein the fulvic acid is an amphiphilic surfactant that emulsifies the oil and water components to keep them in suspension.

2. The topical amphiphilic composition in claim 1, wherein the hyaluronic acid is composed of both low molecular weight and high molecular weight sodium hyaluronate are used in a 5:10 ratio.

3. The topical amphiphilic composition in claim 2, wherein the low molecular weight hyaluronic acid is in a range, measured on the Dalton scale, of between 3 and 7 kDa and the high molecular weight hyaluronic acid is in a range between 10,000 and 20,000 kDa.

4. The topical amphiphilic composition in claim 3, wherein the low molecular weight hyaluronic acid is 5 kDa and the high molecular weight hyaluronic acid is 20,000 kDa.

5. The topical amphiphilic composition in claim 4, further comprising Aloe powder.

6. The topical amphiphilic composition in claim 5, wherein the oil is a plant- or animal-based oil.

7. The topical amphiphilic composition in claim 6, wherein the plant- or animal-based oil is at least one of Olive oil, Evening Primrose oil, Sesame oil, Blackseed oil, Currant seed oil, Pomegranate oil, Almond oil, Macadamia oil, Flax seed oil, Sea Buckthorn oil, Coconut oil, Grapeseed oil, and Emu oil.

8. The topical amphiphilic composition in claim 7, further comprising at least one essential oil.

9. The topical amphiphilic composition in claim 8, wherein the essential oil is at least one of Lavandula Augustifolia essential oil, Eucalyptus essential oil, Rosmarinus officinalis (aka Rosemary) essential oil, Myrrh gum essential oil, Juniper essential oil, Cypress essential oil, Bergamot essential oil, Orange essential oil, Frankincense essential oil, Turmeric essential oil, Ginger essential oil, Neroli essential oil, Chamomile essential oil, Marjoram essential oil, Marjoram essential oil, and Lemon essential oil.

10. The topical amphiphilic composition in claim 9, further comprising at least one extract.

11. The topical amphiphilic composition in claim 10, wherein the extract is at least one of Plantago major extract, Calendula officinalis extract, Hamamelis virginiana extract (American Witch-hazel), Pinus Pinaster extract, Ruscus aculeatus extract, Geranium maculatum extract, Gotu Kola (aka Centalla asiatica) extract, Goldenseal extract, and Lemon balm extract.

12. The topical amphiphilic composition in claim 11, further comprising at least one therapeutic substance.

13. The topical amphiphilic composition in claim 12, wherein the therapeutic substance is at least one of Monoatomic Gold, Vitamin E, and Glucosamine Sulfate.

14. A method of creating a topical composition using an amphiphilic emulsion technique comprising the steps of
Creating a lipidophilic phase by
selecting at least one oil;
introducing hyaluronic acid as powdered sodium hyaluronate;
stirring the hylaluronic acid until blended and dissolved;
adding fulvic acid;
stirring the fulvic acid until, blended and dissolved;
Creating an hydrophilic phase by
dissolving choline citrate, as a stabilizer, in water, creating an aqueous solution;
dissolving potassium sorbate into the aqueous solution as a preservative;
dissolving citric acid into the aqueous solution to adjust the pH; and
dissolving sodium silicate in the aqueous solution;
Combining the lipidophilic phase with the hydrophilic phase into an amphiphilic emulsion.

15. The method of creating a topical composition using an amphiphilic emulsion technique of claim 14, wherein the hyaluronic acid consists of low molecular weight hyaluronic acid in a range of between 3 and 7 kDa and a high molecular weight hyaluronic acid is in a range between 10,000 and 20,000 kDa.

16. The method of creating a topical composition using an amphiphilic emulsion technique claim 15, further comprising the step of mixing in Aloe powder.

17. The method of creating a topical composition using an amphiphilic emulsion technique claim 16, further comprising the step of adding an essential oil.

18. The method of creating a topical composition using an amphiphilic emulsion technique claim 16, further comprising the step of adding an extract.

19. The method of creating a topical composition using an amphiphilic emulsion technique of claim 16, further comprising the step of adding a therapeutic substance.

20. A topical composition comprising:
a water-based ortho silicic acid solution;
sodium hyaluronate in powdered form; and
fulvic acid;
wherein the ortho silicic acid is composed of sodium silicate, citric acid, choline citrate, and potassium sorbate;
wherein the ortho silicic acid is dissolved in water;
wherein the hyaluronic acid is composed of both low molecular weight and high molecular weight sodium hyaluronate in a 5:10 ratio; and
wherein the low molecular weight hyaluronic acid is in a range, measured on the Dalton scale, of between 3 and 7 kDa and the high molecular weight hyaluronic acid is in a range between 10,000 and 20,000 kDa.
